(19) United States
(12) Patent Application Publication
(10) Pub. No.: US 2024/0136027 A1
(43) Pub. Date: Apr. 25, 2024

(54) POINT-OF-USE SYSTEM AND METHOD FOR IDENTIFYING COMPONENTS OF AN UNKNOWN DRUG SAMPLE

(71) Applicants: Unity Health Toronto, Toronto (CA); The Regents of the University of California, Oakland, CA (US)

(72) Inventors: Drew HALL, La Jolla, CA (US); Daniel WERB, Toronto (CA); Daniel BERIAULT, Toronto (CA)

(21) Appl. No.: 18/505,980

(22) Filed: Nov. 9, 2023

Related U.S. Application Data
(63) Continuation of application No. PCT/CA2022/050737, filed on May 11, 2022.
(60) Provisional application No. 63/187,875, filed on May 12, 2021.

(51) Int. Cl. G16C 20/70; G01N 27/416

(52) U.S. Cl. CPC G16C 20/70; G01N 27/416

(57) ABSTRACT

A point-of-use method for identifying one or more components of an unknown drug sample includes electrochemically analyzing the unknown drug sample to obtain an electrochemical measurement, identifying the components of the unknown drug sample by inputting the electrochemical measurement into a machine learning model trained to identify the components based on the electrochemical measurement, and outputting a listing of the components. A point-of-use system for identifying one or more components of an unknown drug sample includes an electrochemical analyzer configured to receive a quantity of the unknown drug sample and conduct an electrochemical analysis to obtain an electrochemical measurement, a non-transitory storage memory storing a machine learning model trained to identify the components based on the electrochemical measurement, and one or more processors in communication with the electrochemical analyzer and configured to input the electrochemical measurement into the machine learning model and output a listing of the components.

### CROSS-REFERENCES TO RELATED APPLICATIONS

This application is a continuation of International Application No. PCT/CA2022/050737, filed on May 11, 2022, which claims priority to U.S. Provisional Application No. 63/187,875, filed May 12, 2021. All of the aforementioned patent applications are hereby incorporated by reference in their entireties.

### FIELD

This document relates to harm reduction for users of illicit drugs. More specifically, this document relates to systems and methods for identifying the components of an unknown drug sample.

### SUMMARY

The following summary is intended to introduce the reader to various aspects of the detailed description, but not to define or delimit any invention.

Point-of-use methods for identifying one or more components of an unknown drug sample are disclosed. According to some aspects, a point-of-use method for identifying one or more components of an unknown drug sample includes: a) electrochemically analyzing the unknown drug sample to obtain an electrochemical measurement; b) identifying the one or more components of the unknown drug sample by inputting the electrochemical measurement into a machine learning model trained to identify the one or more components based on the electrochemical measurement; and c) outputting a listing of the one or more components.

In some examples, the method further includes: d) based on the identification of the one or more components, issuing an alert to a distribution list. Step d) can include comparing the one or more components to a listing of one or more expected components in the unknown drug sample, and issuing the alert based on the comparison. The distribution list can be compiled based on geography.

In some examples, the electrochemical measurement includes a voltammogram generated from square wave voltammetry (SWV), differential pulse voltammetry (DPV), and/or cyclic voltammetry (CV). Step a) can include using a potentiostat to obtain the voltammogram.

In some examples, step a) includes inserting a single-use electrochemical sensor into a portable potentiostat, and applying the sample to the single-use electrochemical sensor.

In some examples, step b) is carried out using a portable computing device, and step c) includes displaying the listing of the one or more components on a display of the portable computing device.

In some examples, the machine learning model is further trained to quantify each of the one or more components based on the electrochemical measurement. Step b) can further include quantifying each of the one or more components. Step c) can further include outputting a quantity of each of the one or more components.

In some examples, the one or more components includes an adulterant. In some examples, the one or more components includes a stimulant and/or an opioid. In some examples, the one or more components includes cocaine, levamisole, heroin, morphine, fentanyl, carfentanil, acetaminophen, ketamine, flualprazolam, fentanyl-related substances, etizolam, flubromazepam, flubromazolam, isonitazene, protonitazene, etonitazene, xylazine, caffeine, 3,4-methylenedioxymethamphetamine (MDMA), 3,4-methylenedioxyamphetamine (MDA) and/or glucose. In some examples, the one or more components includes at least one of cocaine, levamisole, heroin, morphine, fentanyl, carfentanil, MDMA, 3,4-methylenedioxyamphetamine (MDA), acetaminophen, ketamine, and/or flualprazolam.

In some examples, step a) includes dissolving a quantity of the unknown drug sample into a solvent to obtain a solution, and electrochemically analyzing an aliquot of the solution to obtain the electrochemical measurement.

In some examples, step a) includes applying a quantity of the unknown drug sample onto a solid-state hydrogel, and electrochemically analyzing the solid-state hydrogel to obtain the electrochemical measurement

In some examples, the machine learning model is trained using labelled data. The labelled data may be collected by mass spectrometry. In some examples, the machine learning model is trained using unlabelled data.

In some examples, the method further includes: after step a), consuming the sample of the drug composition.

Point-of-use systems for identifying one or more components of an unknown drug sample are disclosed. According to some aspects, a point-of-use system for identifying one or more components of an unknown drug sample includes: an electrochemical analyzer configured to receive a quantity of the unknown drug sample and conduct an electrochemical analysis to obtain an electrochemical measurement; a non-transitory storage memory storing a machine learning model trained to identify the one or more components based on the electrochemical measurement; and one or more processors in communication with the electrochemical analyzer and configured to input the electrochemical measurement into the machine learning model and output a listing of the one or more components.

In some examples, the one or more processors is configured to trigger the issuance of an alert to a distribution list. The one or more processors may be configured to compare the one or more components to a listing of one or more expected components in the unknown drug sample, and trigger the issuance of the alert based on the comparison.

In some examples, the electrochemical analyzer is configured to obtain a voltammogram of the unknown drug sample.

In some examples, the electrochemical analyzer includes a portable potentiostat and a single-use electrochemical sensor.

In some examples, the system further includes a display. The one or more processors may be configured to output the listing of the one or more components to the display.

In some examples, the system further includes a portable computing device that includes the non-transitory storage memory and the one or more processors.

In some examples, the machine learning model is further trained to quantify each of the one or more components based on the electrochemical measurement, and the one or more processors is further configured to output a quantity of each the one or more components.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1 is a perspective view of an example system for identifying one or more components of an unknown drug sample;

FIG. 2 is a flowchart of an example method for identifying one or more components of an unknown drug sample;

FIG. 3 is a plot showing overlaid voltammograms from ketamine standards, ranging from 10 μg/mL to 200 μg/mL;

FIG. 4 is a plot showing extracted peak voltage plotted versus concentration and fit with a linear line, for ketamine;

FIG. 5 is a plot showing overlaid voltammograms from levamisole standards, ranging from 10 μg/mL to 200 μg/mL;

FIG. 6 is a plot showing extracted peak voltage plotted versus concentration and fit with a linear line, for levamisole;

FIG. 7 is a plot showing the extracted peak voltage versus peak current for various drug standards;

FIG. 8 is a plot showing the extracted peak voltage versus FWHM for various drug standards;

FIG. 9 is a plot showing the extracted peak voltage versus peak asymmetry for various drug standards;

FIG. 10 shows the extracted peak voltage versus FWHM for standards where drugs have been grouped into opioids, stimulants, and others.

FIG. 11 is a voltammogram for a first drug sample of unknown composition, annotated with a peak detected at 0.91 V;

FIG. 12 is a voltammogram for a second drug sample of unknown composition, annotated with peaks detected at −0.58 V and 0.99 V;

FIG. 13 is a voltammogram for a third drug sample of unknown composition, annotated with a peak detected at 0.85 V; and

FIG. 14 is a representative confusion matrix for classifying drugs into drug categories using an unoptimized neural network.

### DETAILED DESCRIPTION

Various apparatuses or processes or compositions will be described below. No embodiment described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Numerous specific details are set forth in order to provide a thorough understanding of the subject matter described herein. However, it will be understood by those of ordinary skill in the art that the subject matter described herein may be practiced without these specific details. In other instances, well-known methods, procedures and components have not been described in detail so as not to obscure the subject matter described herein. The description is not to be considered as limiting the scope of the subject matter described herein.

The terms “coupled” or “coupling” or “connecting” or “connected” as used herein can have several different meanings depending on the context in which these terms are used. For example, these terms can have a mechanical, electrical or communicative connotation. For further example, these terms can indicate that two or more elements or devices are directly connected our coupled to one another or connected or coupled to one another through one or more intermediate elements or devices via an electrical element, electrical signal, or a mechanical element depending on the particular context.

As used herein, the wording “and/or” is intended to represent an inclusive-or. That is, “X and/or Y” is intended to mean X or Y or both. As a further example, “X, Y, and/or Z” is intended to mean X or Y or Z or any combination thereof. Furthermore, the wording “at least one of A and B” is intended to mean only A, only B, or A and B.

Terms of degree such as “substantially”, “about”, and “approximately” are intended to mean a reasonable amount of deviation of the modified term such that the end result is not significantly changed. These terms of degree may also be construed as including a deviation of the modified term if this deviation would not negate the meaning of the term it modifies.

Any recitation of numerical ranges by endpoints herein includes all numbers and fractions subsumed within that range, including the endpoints (e.g. 1 to 5 includes 1, 1.5, 2, 2.75, 3, 3.90, 4, and 5). It is also to be understood that all numbers and fractions thereof are presumed to be modified by the term “about”, unless expressly stated otherwise.

The systems and methods described herein may be implemented as a combination of hardware or software. In some cases, the systems and methods described herein may be implemented, at least in part, by using one or more computer programs, executing on one or more programmable devices including at least one processing element, and a data storage element (including volatile and non-volatile memory and/or storage elements). These devices may also have at least one input device (e.g. a pushbutton keyboard, mouse, a touchscreen, and the like), and at least one output device (e.g. a display screen, a printer, a wireless radio, and the like) depending on the nature of the device.

Some elements that are used to implement at least part of the systems and methods described herein may be implemented via software that is written in a high-level procedural language such as object-oriented programming. Accordingly, the program code may be written in any suitable programming language, such as Python or C. Alternatively or in addition thereto, some of these elements implemented via software may be written in assembly language, machine language, or firmware as needed. In either case, the language may be a compiled or interpreted language.

At least some of these software programs may be stored on a storage media (e.g. a computer readable medium such as, but not limited to, ROM, magnetic disk, optical disc) or a device that is readable by a general or special purpose programmable device. The software program code, when read by the programmable device, configures the programmable device to operate in a new, specific and predefined manner in order to perform at least one of the methods described herein.

Furthermore, at least some of the programs associated with the systems and methods described herein may be capable of being distributed in a computer program product including a computer readable medium that bears computer usable instructions for one or more processors. The medium may be provided in various forms, including non-transitory forms such as, but not limited to, one or more diskettes, compact disks, tapes, chips, and magnetic and electronic storage. Alternatively, the medium may be transitory in nature such as, but not limited to, wire-line transmissions, satellite transmissions, internet transmissions (e.g. downloads), media, digital and analog signals, and the like. The computer useable instructions may also be in various formats, including compiled and non-compiled code.

Generally disclosed herein are systems and methods for identifying one or more components of a drug sample, such as an unknown and/or illicit drug sample. For example, the systems and methods may allow for users of illicit drugs to confirm the components of a drug sample (e.g. to confirm that a substance that is believed to be heroin is indeed heroin), or to test for the presence of adulterants in a drug sample (e.g. to test for the presence of fentanyl in a substance that is expected to contain only heroin). Thus, the system and method may be used for harm reduction.

In general, the system can include an electrochemical analyzer (e.g. a potentiostat coupled with an electrochemical sensor) that receives a quantity of a drug sample and conducts an electrochemical analysis to obtain an electrochemical measurement (e.g. a voltammogram); a non-transitory storage memory that stores a machine learning model trained to identify the component(s) of the drug sample based on the electrochemical measurement; and one or more processors that are in communication with the electrochemical analyzer and that input the electrochemical measurement into the machine learning model and output a listing of the component(s). In general, the method can include electrochemically analyzing a drug sample to obtain an electrochemical measurement; identifying the component(s) of the drug sample by inputting the electrochemical measurement into a machine learning model trained to identify the component(s) based on the electrochemical measurement; and outputting a listing of the component(s).

As used herein, the term “drug sample” refers to a substance that contains one or more drugs, such psychoactive compounds (e.g. opioids, stimulants, hallucinogens, and the like) and/or non-psychoactive compounds (e.g. acetaminophen), and optionally additional compounds (e.g. glucose). The term “illicit” indicates that the drug sample is being used for non-medical reasons (e.g. recreation), and/or is illegal to possess (either outright or without a prescription) in the jurisdiction in which the drug sample is being used. The phrase “illicit drug sample” is interchangeable with the phrase “street drug”. The term “unknown” indicates that the component(s) of the drug sample are not known to the user, and/or that the user desires to obtain information regarding the component(s) of the drug sample. While the systems and methods described herein may be usable with various drug samples, they may be particularly beneficial for use with illicit drug samples, which are often subject to adulteration, and which are often subject to uncertainty in their composition (i.e. the composition of illicit drug samples is often unknown).

Advantageously, the system described herein may be a “point-of-use” system. That is, the system may be used in the field (such as at a safe injection site, outdoors, or in a private residence), without the need for complicated and/or expensive laboratory equipment (such as a mass spectrometer), and may provide results on-site and in a short period of time (e.g. within seconds or minutes). Likewise, the method may be a “point-of-use” method.

Advantageously, the system described herein may be portable. For example, the system can include a portable potentiostat and a supply of single-use electrochemical sensors. The portable potentiostat can be in communication (e.g. wireless communication) with a portable computing device, such as but not limited to a user's smartphone, tablet, smartwatch, or laptop computer. The portable computing device can store the machine learning model, input the electrochemical measurement into the machine learning model, and output a listing of the components of the drug sample (e.g. on a display of the portable computing device).

Advantageously, the system and method may be user-friendly. For example, the system and method may be used by illicit drug users themselves, or by staff of a harm reduction site, without the need for in-depth training, expertise, and/or experience (e.g. experience in laboratory methods). A mobile app can be provided that facilitates use.

Advantageously, the system and method may require only 1 mg of a drug sample, and therefore, will not consume a significant proportion of a single use drug of 50-100 mg in total.

Advantageously, the system and method can reduce harm not only to the individual in possession of the drug sample, but also to the broader community. For example, if an individual tests a drug sample that is expected to contain a given drug (e.g. heroin), and determines that the drug sample contains an adulterant (e.g. fentanyl), the system may issue an alert to other users within the same geographical region, alerting them that heroin bought within that geographical region contains fentanyl. This may be achieved by the mobile app mentioned above. That is, the mobile app may be used by individuals that are testing drug samples, and by other individuals who may benefit from having access to the results of the test.

Advantageously, in addition to identifying the component(s) of a drug sample, the system and method may also quantify each component.

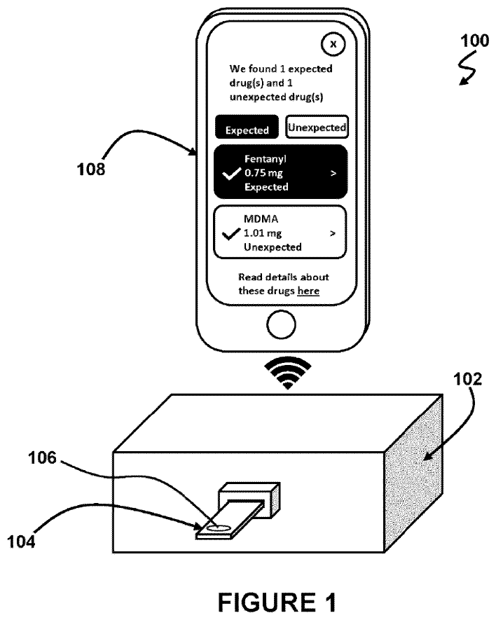

Referring now to FIG. 1, an example system 100 is shown. The system 100 can be used to identify one or more components of a drug sample, such as an illicit and/or unknown drug sample. Such components may include, for example, cocaine, levamisole, heroin, morphine, fentanyl, carfentanil, 3,4-methylenedioxymethamphetamine (MDMA), 3,4-methylenedioxyamphetamine (MDA), acetaminophen, ketamine, and/or flualprazolam. It has been determined that the aforementioned components can be identified and quantified with the system 100. Such components may further include fentanyl-related substances (i.e. fentanyl analogs or derivatives, such as acetyl fentanyl), etizolam, flubromazepam, flubromazolam, isonitazene, protonitazene, etonitazene, xylazine, caffeine, 3,4-methylenedioxymethamphetamine (MDMA), amphetamine, codeine, 6-monoacetylmorphine (6-MAM), fructose, glucose, and theophylline. It is believed that the aforementioned components will be identifiable and quantifiable with the system 100 with further training of the machine learning model described below. It is further believed that as new illicit drugs come into use, at least some of these new drugs may be identifiable and quantifiable with the system 100.

As described above, the components identified by the system 100 may be expected components, or unexpected components (i.e. adulterants). Such adulterants may themselves be drugs, or may be other compounds (e.g. glucose). For example, as shown in FIG. 1, an individual may be in possession of an illicit drug sample that is believed to be pure fentanyl. The system 100 can be used to identify adulterants such as 3,4-methyl enedioxymethamphetamine (MDMA) in the illicit drug sample. Alternatively, the system can indicate that no detectable adulterants were identified in the illicit drug sample.

Referring still to FIG. 1, the system 100 generally includes an electrochemical analyzer that is configured to receive a quantity of the drug sample (e.g. an aliquot of a solution of the drug sample in a buffer), and conduct an electrochemical analysis of the drug sample to obtain an electrochemical measurement. In the example shown, the electrochemical analyzer includes a potentiostat 102, and an electrochemical sensor 104 (e.g. a screen-printed electrode that includes a working electrode 106, a counter electrode (not shown), and a reference electrode (not shown)). The potentiostat 102 may be portable (e.g. may be battery powered and may be relatively small and lightweight), and the electrochemical sensor 104 may be a single-use sensor. As will be described in further detail below, in use, the electrochemical sensor 104 can be inserted into the potentiostat 102, and a quantity of a drug sample (e.g. an aliquot of a solution of the drug sample in a buffer) can be applied to the working electrode 106 of the electrochemical sensor 104. A voltammogram can then be generated for the drug sample, for example using square wave voltammetry (SWV), differential pulse voltammetry (DPV), and/or cyclic voltammetry (CV).

Referring still to FIG. 1, the system 100 further includes a non-transitory storage memory (not shown), and one or more processors (not shown). The non-transitory storage memory stores a machine learning model that is trained to identify the components of the drug sample based on the electrochemical measurement (i.e. the voltammogram), and to quantify each of the components based on the electrochemical measurement. The processor(s) is/are in communication with the electrochemical analyzer (e.g. wireless communication) and is/are configured to input the electrochemical measurement into the machine learning model and output a listing of the one or more components and quantity of each of the components. In the example shown, the system includes a portable computing device in the form of a smart phone 108, which includes the non-transitory storage memory and the processor(s). The smart phone 108 further includes a display, and the processor is configured to output the listing of components and the quantity of each component to the display, as shown in FIG. 1. In an alternative example, the non-transitory storage memory, processor(s), and/or display can be included in the potentiostat 102.

Various types of machine learning models may be used to identify the components of the drug sample. For example, a deep learning neural network model may be used to identify the components of the drug sample. Alternatively, a different type of machine learning model may be used to identify the components of the drug sample, such as a neural network, a gradient boosted decision tree, a support vector machine, and so on. Furthermore, combinations of the above types of machine learning models may be used (e.g. fentanyl may be detected with a neural network, while morphine may be detected with a support vector machine). Furthermore, layers of the above types of machine learning models may be used (e.g. where a plurality of binary classifiers are combined with a second machine learning model).

The machine learning model may employ one or more of feature extraction, label encoding, feature scaling, stratified shuffle split, hyperparameter optimization, or performance assessment.

The machine learning model can be trained using training data that may be labelled data or non-labelled data. Labelled data can be obtained, for example, using mass spectrometry (as described in further detail below). The machine learning model can be trained using the training data, in order to determine a correlation between the electrochemical measurement and the identity and quantity of the components of the drug sample. The machine learning model can then be optimized to maximize the correlation.

Notably, it has been determined that the machine learning model can differentiate between components that have very similar peaks on a voltammogram. For example, on a voltammogram, carfentanil and fentanyl have a signature peak at similar voltages. The machine learning model can nevertheless differentiate between the two substances using additional features such as the full width at half maximum (FWHM), peak asymmetry, etc.

The machine learning model may further be configured to adjust its output based on the identified components. For example, in some instances, it may not be possible or feasible to differentiate between two or more specific substances. Particularly, it has been determined that in a voltammogram, morphine has a secondary peak that overlaps with the peak for fentanyl, and crosses over into the peak for heroin at higher concentrations. As such, with mixtures of morphine and fentanyl or morphine and heroin, the machine learning model may not be able to accurately identify the fentanyl or the heroin. Accordingly, the machine learning model may be configured such that it does not attempt to identify fentanyl and heroin if morphine is identified in the drug sample.

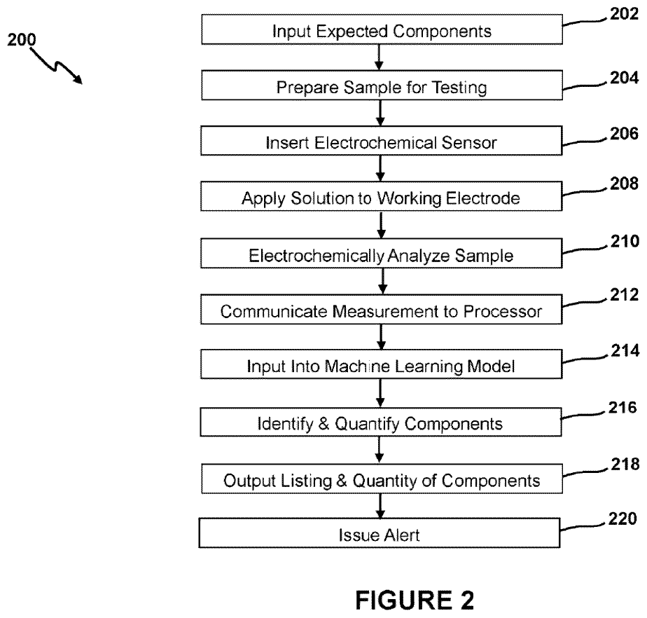

Referring now to FIG. 2, an example method 200 is shown. The method can be used to identify one or more components of a drug sample, such as an unknown and/or illicit drug sample. The method will be described with reference to the system 100; however, the method is not limited to use with the system 100, and the system 100 is not limited to use according to the described method. Furthermore, for ease of understanding, terms such as “next”, “first”, or “then” may be used with regards to the order of the steps of the method; however, the method is not limited to any particular order of steps, unless expressly stated as such.

As a first step, using the portable computing device (e.g. smartphone 108), a user that is in possession of a drug sample can input the expected components of the drug sample (step 202). This can be done using the mobile app described above, via the user interface shown in FIG. 1. For example, if a user believes that they are in possession of fentanyl, the user may select “fentanyl” from a list of possible components. Alternatively, the step of inputting the expected components can be omitted, for example if the user does not have an expectation of the components of the sample.

As a next step, the drug sample can be prepared for testing (step 204). For example, using a standardized scoop, a quantity of the drug sample (e.g. 1 mg of powdered, crushed crystalline, or liquid phase drug sample) can be dissolved into a solvent of a standardized pH. The solvent can optionally be provided or sold with the system. The solvent can be, for example, phosphate buffered saline (PBS).

In an alternative example, rather than using a solvent, the electrochemical sensor may be provided with a solid-state hydrogel on the working electrode. The hydrogel can have a standardized pH. A quantity of the drug sample (e.g. 1 mg of powdered, crushed crystalline, or liquid phase drug sample) can then be applied directly to the hydrogel.

As a next step, the electrochemical sensor 104 can be inserted into the potentiostat 102 (step 206), and an aliquot of the solution prepared in step 204 can be applied to the working electrode 106 of the electrochemical sensor 104 (step 208), for example using a dropper or pipette. Alternatively, the electrochemical sensor 104 can be dipped into the solution prepared in step 204, to immerse the working electrode 106 in the solution and thereby apply an aliquot of the solution to the working electrode 106, and then the electrochemical sensor 104 can be inserted into the potentiostat 102.

As a next step, either automatically or on receipt of a command from the user (e.g. the pressing of a start button by the user), the drug sample can be electrochemically analyzed by the potentiostat 102 (step 210), to obtain an electrochemical measurement of the drug sample (i.e. a voltammogram, in the example shown).

As a next step, the electrochemical measurement can be communicated to the processor of the portable computing device (e.g. wirelessly, optionally via Bluetooth®, or via a wired connection, such as USB) (step 212), which can in turn input the electrochemical measurement into the machine learning model stored on the portable computing device (step 214). Based on the electrochemical measurement, the machine learning model can then identify and optionally quantify the components of the drug sample (step 216). A listing of the components, as well as the quantity of each component, can then be output (step 218). For example, a listing of the components and the quantity of each component can be displayed on a display of the portable computing device (as shown in FIG. 1).

As mentioned above, the method can provide results within a short time frame. For example, steps 202 to 218 may be completed in under 3 minutes.

Optionally, based on the identification of the components, an alert can be issued to a distribution list (step 220). For example, the identified components can be compared to the expected components, which were input in step 202. If the identified components and the expected components do not match, an alert can be issued to a distribution list. The distribution list can be compiled based on geography, so that users within the same geographical area can be made aware that illicit drugs obtained within a given geographical area may contain adulterants. The alert can be issued, for example, to all users of the app within a given geographical area.

Optionally, the prepared drug sample (i.e. the solution prepared in step 204, or the hydrogel described above) can be consumed after the test is complete.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

### EXAMPLES

A panel of drug standards and a panel of illicit drug samples (obtained via Toronto's Drug Checking service) were tested using the systems and methods described herein. In total, 249 drug standards were tested, and 819 illicit drug samples were tested. Drug standards included morphine, MDMA, levamisole, acetaminophen, heroin, fentanyl, carfentanil, ketamine, and cocaine. As described above, it was determined that cocaine, levamisole, heroin, morphine, fentanyl, carfentanil, MDMA, MDA, acetaminophen, ketamine, and flualprazolam can be identified and quantified with the systems and methods described herein. Furthermore, is believed that with further training of the machine learning model, additional components may be identifiable and quantifiable.

Sample Collection

Training Data Collection. Drug standards (often supplied in methanol) including morphine, MDMA, levamisole, acetaminophen, heroin, fentanyl, carfentanil, ketamine, and cocaine were diluted in phosphate-buffered saline (PBS), pH 7, to final concentrations ranging from 10 μg/mL to 400 μg/mL. 100 μL of the sample was placed on a screen-printed electrode (SPE), which was inserted into a potentiostat. Voltammograms were recorded with the potentiostat using differential pulse voltammetry (DPV) from −0.6 to 1.3 V, 10 mV step size, 25 mV pulse for 50 ms, and a scan rate of 50 mV/s.

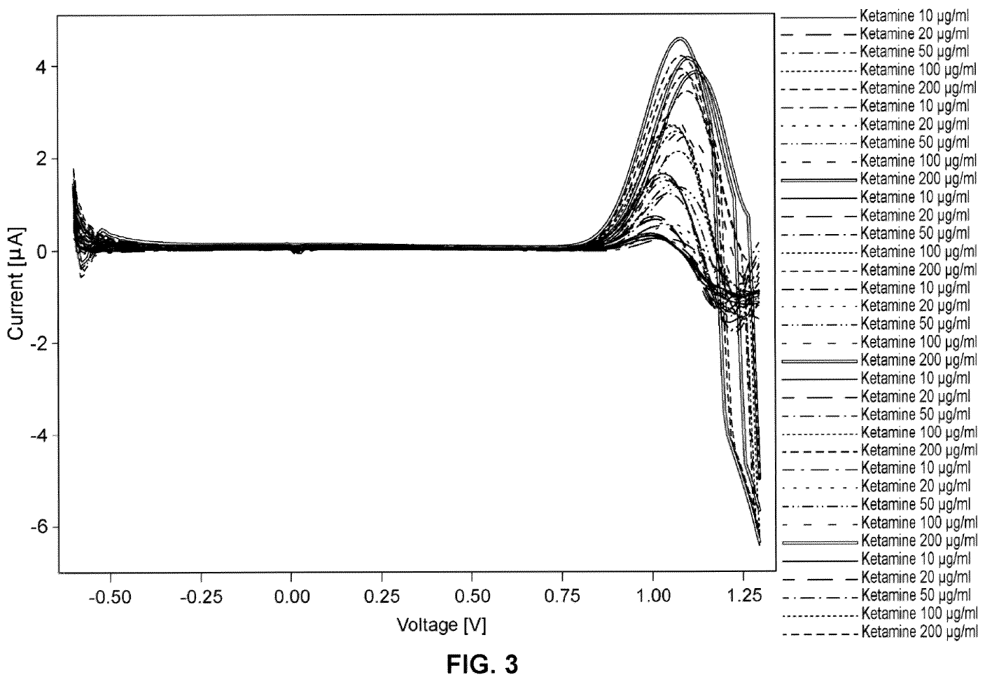

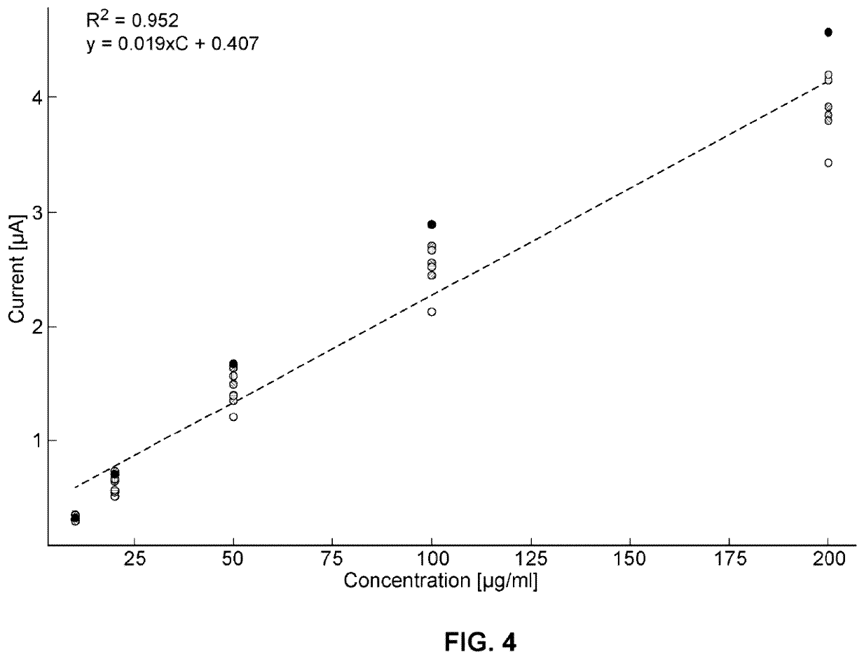

FIG. 3 shows overlaid voltammograms from ketamine standards, ranging from 10 μg/mL to 200 μg/mL, where each line represents one measurement. FIG. 4 shows extracted peak voltage plotted versus concentration and fit with a linear line, for ketamine.

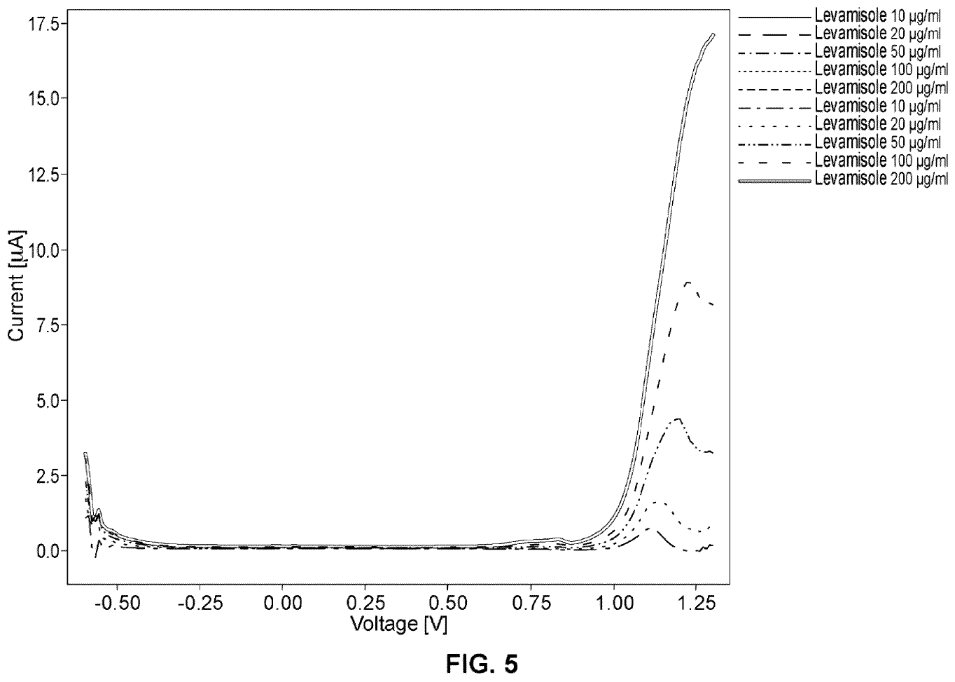

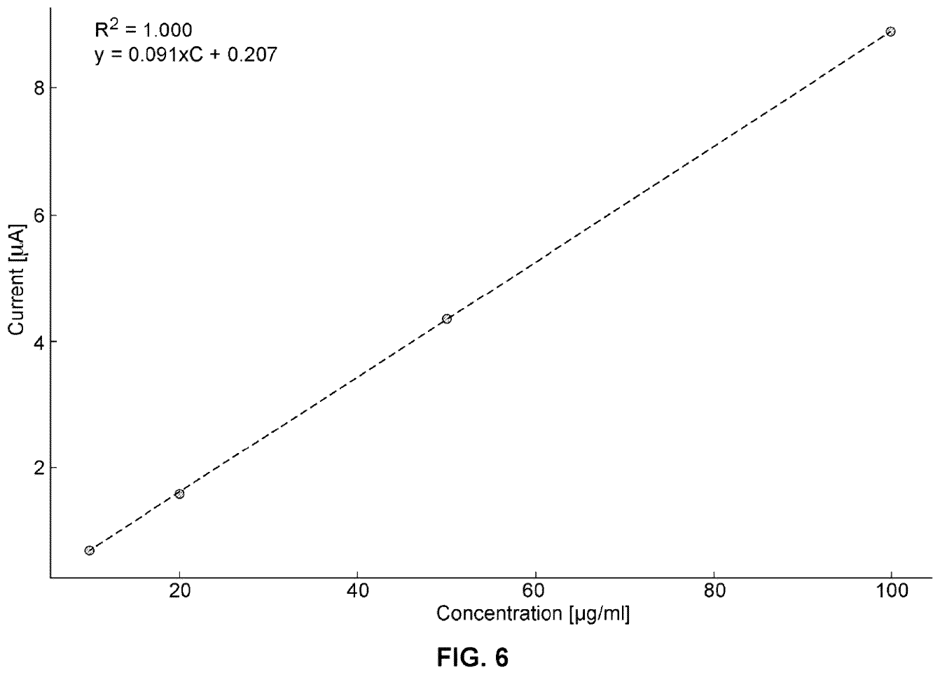

FIG. 5 shows overlaid voltammograms from levamisole standards, ranging from 10 μg/mL to 200 μg/mL, where each line represents one measurement. FIG. 6 shows extracted peak voltage plotted versus concentration and fit with a linear line, for levamisole.

For brevity, voltammograms and plots of extracted peak voltage versus peak current are not shown for all drug standards. However, Table 1 shows a summary of the calibration curves for the various drug standards. MinV/MaxV are the minimum and maximum voltage for the peak.

TABLE 1

| | Intercept | Slope | | MinV | MaxV |
|---|---|---|---|---|---|
| Drug | [μA] | [μA/(μg/ml)] | R² | [V] | [V] |
| Morphine | 0.42 | 0.01 | 0.98 | 0.39 | 0.41 |
| MDMA | 0.43 | 0.08 | 0.94 | 1.02 | 1.07 |
| Levamisole | −0.21 | 0.09 | 1.00 | 1.11 | 1.23 |
| Acetaminophen | 2.41 | 0.09 | 0.94 | 0.32 | 0.36 |
| Heroin | 0.12 | 0.03 | 0.91 | 0.85 | 0.93 |
| Fentanyl | 0.64 | 0.03 | 0.92 | 0.79 | 0.90 |
| Carfentanil | 0.71 | 0.05 | 0.97 | 0.80 | 0.92 |
| Ketamine | 0.41 | 0.02 | 0.95 | 0.99 | 1.12 |
| Cocaine | 0.26 | 0.02 | 0.94 | 0.99 | 1.0 |

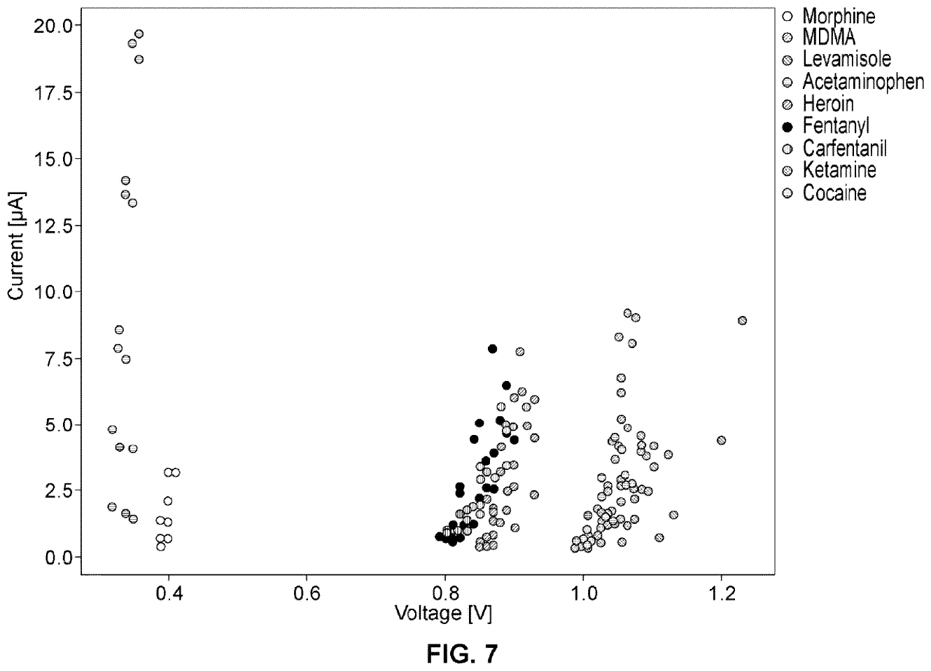

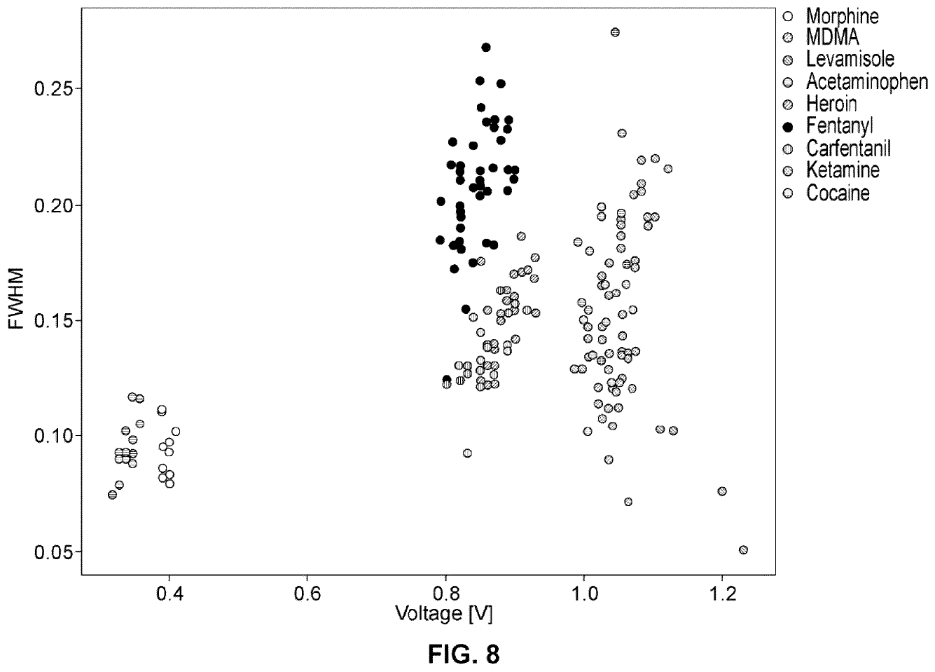

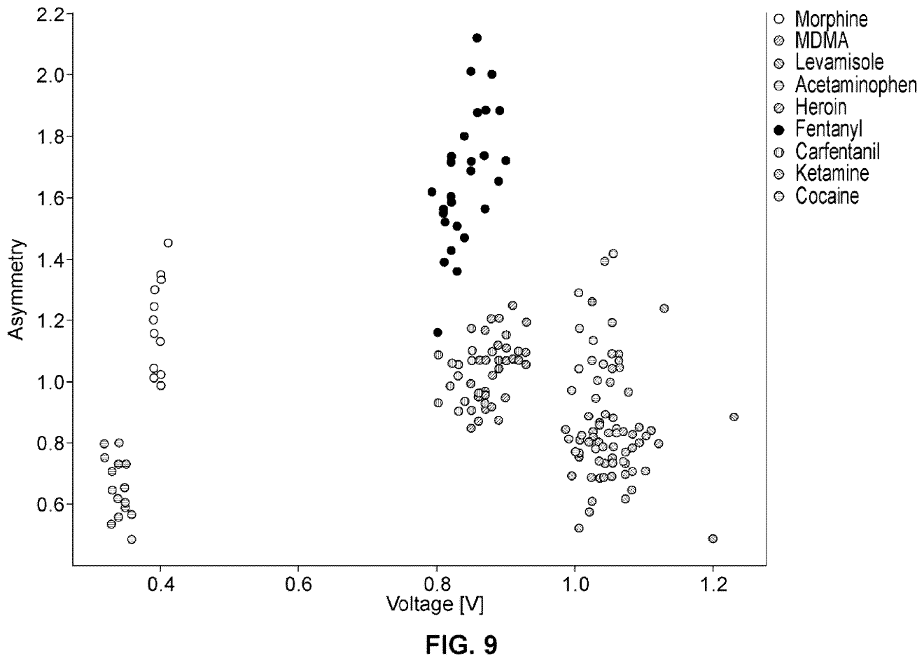

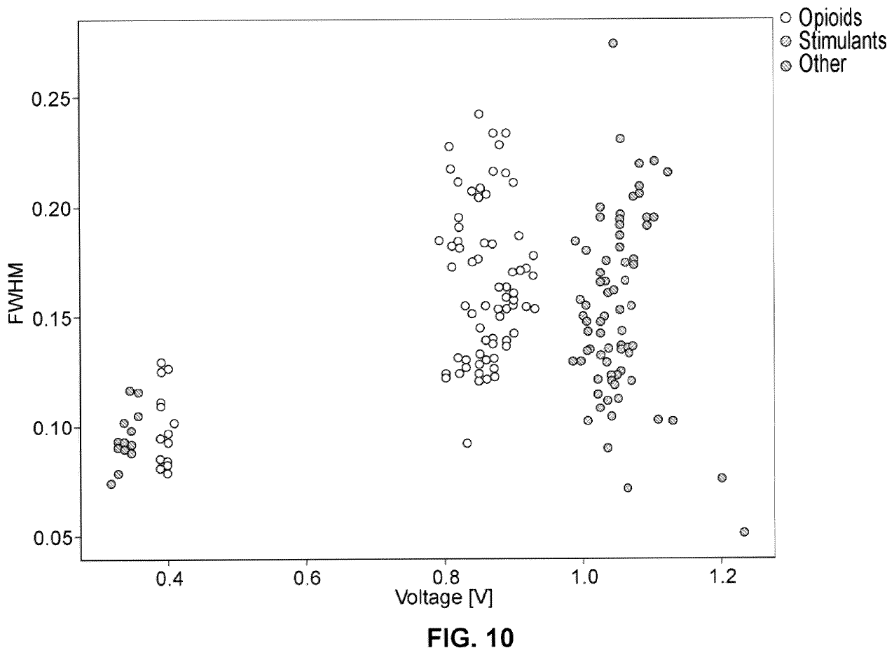

Furthermore, FIG. 7 shows the extracted peak voltage plotted versus peak current for the various drug standards. FIG. 8 shows the extracted peak voltage plotted versus FWHM for the various drug standards. FIG. 9 shows the extracted peak voltage plotted versus peak asymmetry for the various drug standards. FIG. 10 shows the extracted peak voltage versus FWHM for standards where drugs have been grouped into opioids, stimulants, and others.

Illicit Drug Sample Collection and Validation. Illicit drug samples (supplied as powder or paraphernalia scrapings) were provided in various amounts from the Toronto Drug Checking Service. 10 mg of each illicit drug sample was diluted in 1 mL methanol (10 mg/mL) and run on a gas chromatography-mass spectrometer (GC-MS) (Agilent GC6890N-MS 5975 system ISO platform). 50 μL of the solution was then diluted in 2 mL of PBS. 100 μL of the diluted solution was applied to an SPE placed in the potentiostat Voltammograms were recorded with the potentiostat using the IPPV parameters previously described.

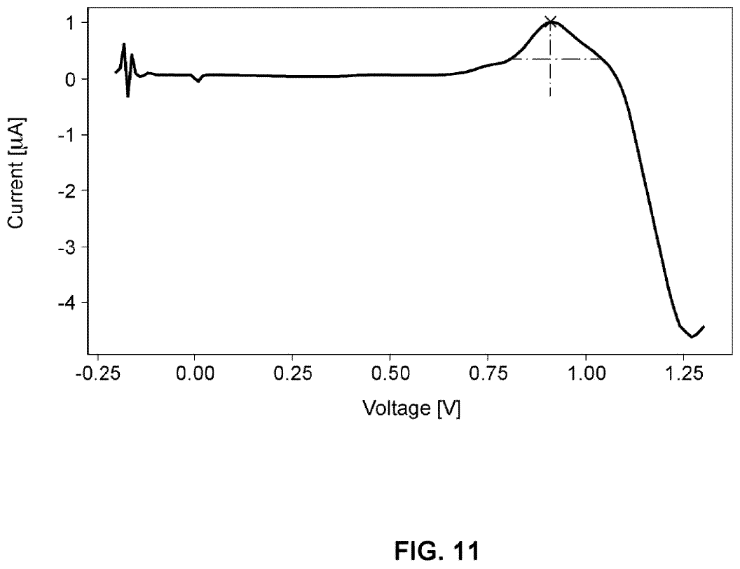

FIG. 11 shows a voltammogram for a first illicit drug sample of unknown composition, annotated with a peak detected at 0.91 V.

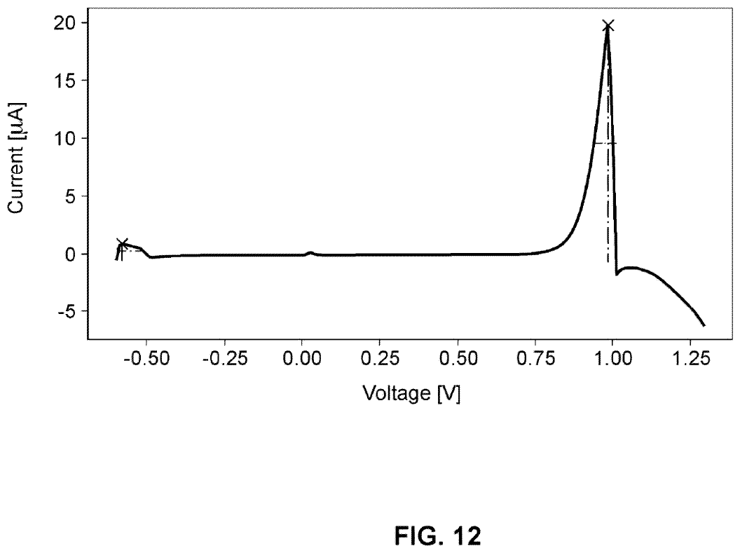

FIG. 12 shows a voltammogram for a second illicit drug sample of unknown composition, annotated with peaks detected at −0.58V and 0.99V.

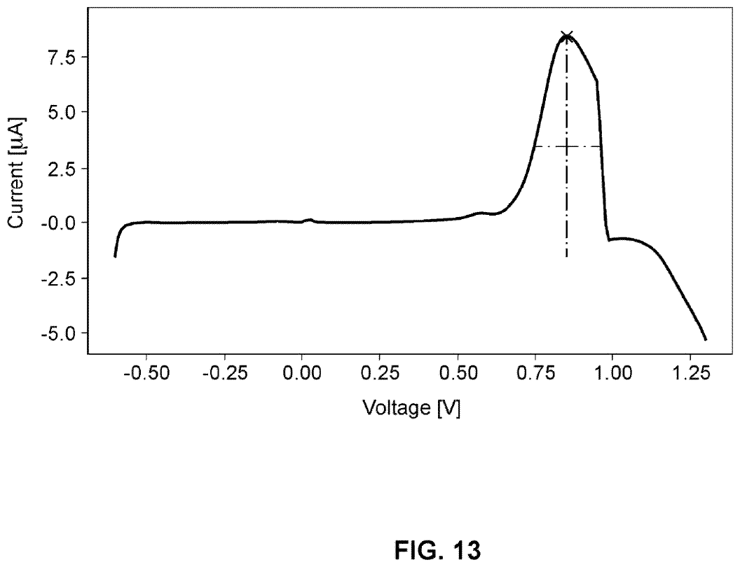

FIG. 13 shows a voltammogram for a third illicit drug sample of unknown composition, annotated with a peak detected at 0.85V.

For brevity, voltammograms for all illicit drug samples are not shown.

Machine Learning Classification

Feature Extraction. A peak finding algorithm was used to find all peaks in the voltammograms. Peaks with low amplitudes (<0.3 μA and/or narrow widths (<5 mV) were discarded as they were deemed likely to be from noise/interference and not underlying signals. Several additional properties were extracted for each peak, including full width at half-max (FWHM), asymmetry, and area under the peak. These features as well as voltage and peak amplitude were used for classification inputs to the machine learning algorithms. Custom feature extraction algorithms were written in Python.

Table 2 shows a summary of the extracted feature ranges for the various drug standards.

TABLE 2

| | Peak Voltage | Peak Current | FWHM | Asymmetry |
|---|---|---|---|---|
| Drug | (V) | (uA) | (a.u.) | (unitless) |
| Cocaine | 0.99-1.07 | 0.42-4.48 | 0.10-0.27 | 0.74-1.41 |
| Heroin | 0.85-0.93 | 0.38-7.74 | 0.12-0.19 | 0.85-1.25 |
| Morphine | 0.39-0.41 | 0.38-3.18 | 0.08-0.11 | 0.99-1.46 |
| Fentanyl | 0.79-0.90 | 0.59-7.80 | 0.12-0.24 | 1.16-2.12 |
| MDMA | 1.02-1.07 | 0.74-9.13 | 0.07-0.14 | 0.57-1.09 |
| Acetaminophen | 0.32-0.36 | 1.42-19.68 | 0.07-0.12 | 0.48-0.80 |
| Ketamine | 0.99-1.12 | 0.30-4.14 | 0.13-0.22 | 0.61-0.85 |
| Carfentanil | 0.80-0.92 | 0.88-5.67 | 0.09-0.16 | 0.91-1.17 |
| Levamisole | 1.11-1.23 | 0.72-8.89 | 0.05-0.10 | 0.49-1.24 |

Table 3 shows a summary of the peaks and features found in the first illicit drug sample (of FIG. 11).

TABLE 3

| Peak Voltage | Peak Current | FWHM | Asymmetry |
|---|---|---|---|
| (V) | (uA) | (a.u.) | (unitless) |
| 0.91 | 1.01 | 0.24 | 1.28 |

Table 4 shows a summary of the peaks and features found in the second illicit drug sample (of FIG. 12).

TABLE 4

| Peak Voltage | Peak Current | FWHM | Asymmetry |
|---|---|---|---|
| (V) | (uA) | (a.u.) | (unitless) |
| −0.58 | 0.84 | 0.08 | 4.85 |
| 0.99 | 19.77 | 0.06 | 0.39 |

Table 5 shows a summary of the peaks and features found in the third illicit drug sample (of FIG. 13).

TABLE 5

| Peak Voltage | Peak Current | FWHM | Asymmetry |
|---|---|---|---|
| (V) | (uA) | (a.u.) | (unitless) |
| 0.85 | 8.42 | 0.21 | 1.06 |

Model Training and Classification. Standards data were split into training and test validation datasets with repeated stratified k-fold (k=5) partitioning. Features were scaled and then fit using various models (e.g., support vector machine (SVM), k-nearest neighbors (FINN), random forest (RF), etc.). With cross-validation, model performance was evaluated based on the F1-score (the harmonic mean of the precision and recall). Hyperparameters for each model were adjusted and optimized to maximize performance. Models were either set up as an array of binary classifiers (e.g., Yes/No fentanyl, Yes/No cocaine, etc.) or multiclass (e.g., Cocaine, Heroin, and Fentanyl). For binary classification, under-sampling or artificial oversampling (i.e., Synthetic Minority Over-sampling Technique—SMOTE) were used to combat class imbalance. An unknown state was added and trained with off-target data (e.g., acetaminophen, caffeine, etc.) for multiclass classification. Performance was assessed based on the F1-score and confusion matrices, Classification algorithms were implemented in Python using the sci-kit learn library.

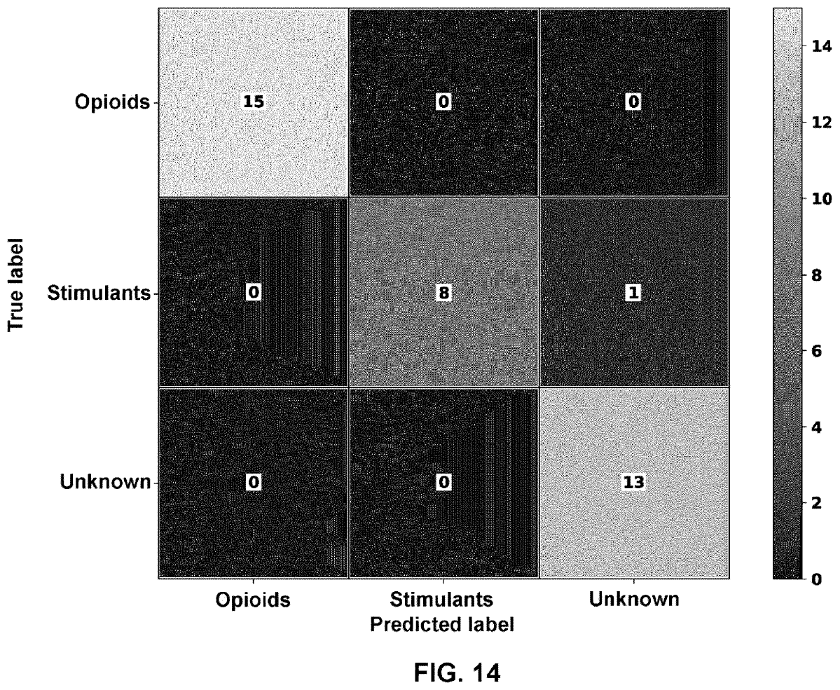

FIG. 14 shows a representative confusion matrix for classifying drugs into drug categories using an unoptimized neural network.

Neural Network Classification

Resampling. Voltammograms from training samples and illicit drug samples were cropped to 0.2-1.2 V and resampled such that all data had the same voltage points as some data were collected with different DPV parameters, specifically a wider scan range. Resampling code was implemented in Python using the numpy library.

Model Training and Classification. Resampled voltammograms were scaled and used directly as the inputs to the neural network without any other post-processing. Models were constructed with dense layers, dropout layers, convolutional layers, and pooling layers as either artificial neural networks (ANN) or convolutional neural networks (CNN). The number of inputs corresponded to the voltages of the resampled voltammograrns, and the outputs were binary, with one output per class. Standard data were split into training and test validation datasets with repeated stratified k-fold (k=5) partitioning. The model was fit using a categorical cross-entropy loss function and evaluated based on the F1-score. Hyperparameters for each model were adjusted and optimized to maximize performance. Classification algorithms were implemented in Python using keras and tensorflow libraries.

Table 6 shows a summary of multiclass random forest prediction and the true value of the first illicit drug sample. The unknown sample was correctly identified as fentanyl, with no other adulterants detected.

TABLE 6

| | | | | | | |
|---|---|---|---|---|---|---|
| Acetamin- | Carfen- | | Fen- | Her- | | Mor- |
| ophen | tanil | Cocaine | tanyl | oin | MDMA | phine |

| | |
|---|---|
| Predicted | 1 |
| True | 1 |

Table 7 shows a summary of multiclass random forest prediction and the true value of the second illicit drug sample. The unknown sample was correctly identified as MDMA, with no other adulterants detected.

TABLE 7

| | | | | | | |
|---|---|---|---|---|---|---|
| Acetamin- | Carfen- | | Fen- | Her- | | Mor- |
| ophen | tanil | Cocaine | tanyl | oin | MDMA | phine |

| | |
|---|---|
| Predicted | 1 |
| True | 1 |

Table 8 shows a summary of multiclass random forest prediction and the true value of the third illicit drug sample. The unknown sample was correctly identified as containing fentanyl, but missed detecting cocaine. It is believed that with further training, the machine learning model will be able to identify cocaine in samples containing both fentanyl and cocaine.

TABLE 8

| | | | | | | |
|---|---|---|---|---|---|---|
| Acetamin- | Carfen- | | Fen- | Her- | | Mor- |
| ophen | tanil | Cocaine | tanyl | oin | MDMA | phine |

| | | |
|---|---|---|
| Predicted | | 1 |
| True | 1 | 1 |

1. A point-of-use method for identifying one or more components of an unknown drug sample, comprising:
a. electrochemically analyzing the unknown drug sample to obtain an electrochemical measurement;
b. identifying the one or more components of the unknown drug sample by inputting the electrochemical measurement into a machine learning model trained to identify the one or more components based on the electrochemical measurement; and
c. outputting a listing of the one or more components.

2. The method of claim 1, further comprising:
d. based on the identification of the one or more components, issuing an alert to a distribution list.

3. The method of claim 2 wherein step d. comprises comparing the one or more components to a listing of one or more expected components in the unknown drug sample, and issuing the alert based on the comparison.

4. The method of claim 2, wherein the distribution list is compiled based on geography.

5. The method of claim 1, wherein the electrochemical measurement comprises a voltammogram generated from square wave voltammetry (SWV), differential pulse voltammetry (DPV), and/or cyclic voltammetry (CV), and step a. comprises using a potentiostat to obtain the voltammogram.

6. The method of claim 1, wherein step a. comprises:
inserting a single-use electrochemical sensor into a portable potentiostat; and
applying the sample to the single-use electrochemical sensor.

7. The method of claim 1, wherein step b. is carried out using a portable computing device, and step c. comprises displaying the listing of the one or more components on a display of the portable computing device.

8. The method of claim 1, wherein:
the machine learning model is further trained quantify each of the one or more components based on the electrochemical measurement;
step b. further comprises quantifying each of the one or more components; and
step c. further comprises outputting a quantity of each of the one or more components.

9. The method of claim 1, wherein the one or more components comprises an adulterant.

10. The method of claim 1, wherein the one or more components comprises a stimulant and/or an opioid.

11. The method of claim 1, wherein the one or more components comprises at least one of cocaine, levamisole, heroin, morphine, fentanyl, carfentanil, acetaminophen, ketamine, flualprazolam, fentanyl-related substances etizolam, flubromazepam, flubromazolam, isonitazene, protonitazene, etonitazene, xylazine, caffeine, 3,4-methylenedioxymethamphetamine (MDMA), 3,4-methylenedioxyamphetamine (MDA), and glucose.

12. The method of claim 1, wherein the one or more components comprises at least one of cocaine, levamisole, heroin, morphine, fentanyl, carfentanil, MDMA, MDA, acetaminophen, ketamine, and/or flualprazolam.

13. The method of claim 1, wherein step a. comprises dissolving a quantity of the unknown drug sample into a solvent to obtain a solution, and electrochemically analyzing an aliquot of the solution to obtain the electrochemical measurement

14. The method of claim 1, wherein step a. comprises applying a quantity of the unknown drug sample onto a solid-state hydrogel on the working electrode, and electrochemically analyzing the solid-state hydrogel to obtain the electrochemical measurement

15. The method of claim 1, wherein the machine learning model is trained using labelled data.

16. The method of claim 15, wherein the labelled data collected by mass spectrometry.

17. The method of claim 1, wherein the machine learning model is trained using unlabelled data.

18. The method of claim 1, further comprising, after step a., consuming the sample of the drug composition.

19. A point-of-use system for identifying one or more components of an unknown drug sample, comprising:
an electrochemical analyzer configured to receive a quantity of the unknown drug sample and conduct an electrochemical analysis to obtain an electrochemical measurement;
a non-transitory storage memory storing a machine learning model trained to identify the one or more components based on the electrochemical measurement; and
one or more processors in communication with the electrochemical analyzer and configured to input the electrochemical measurement into the machine learning model and output a listing of the one or more components.

20. The system of claim 19, wherein the one or more processors is configured to trigger the issuance of an alert to a distribution list.

21. The system of claim 20, wherein the one or more processors is configured to compare the one or more components to a listing of one or more expected components in the unknown drug sample, and trigger the issuance of the alert based on the comparison.

22. The system of claim 19, wherein the electrochemical analyzer is configured to obtain a voltammogram of the unknown drug sample.

23. The system claim 19, wherein the electrochemical analyzer comprises a portable potentiostat and a single-use electrochemical sensor.

24. The system of claim 19, further comprising a display, wherein the one or more processors is configured to output the listing of the one or more components to the display.

25. The system of claim 19, further comprising a portable computing device that includes the non-transitory storage memory and the one or more processors.

26. The system of claim 19, wherein the machine learning model is further trained to quantify each of the one or more components based on the electrochemical measurement, and the one or more processors is further configured to output a quantity of each the one or more components.
